(19) United States
(12) Patent Application Publication
(10) Pub. No.: US 2026/0315558 A1
(43) Pub. Date: Oct. 15, 2026

(54) PROBIOTICS METABOLITE BLEND FOR FOOD AND BEVERAGE APPLICATIONS

(71) Applicants: Sasikanth Chemalamudi, San Fernando (CL); Priyanka Srinivas, San Fernando (CL)

(72) Inventors: Sasikanth Chemalamudi, San Fernando (CL); Priyanka Srinivas, San Fernando (CL)

(21) Appl. No.: 19/178,925

(22) Filed: Apr. 15, 2025

(51) Int. Cl. A61K 36/064; A23L 2/38; A23L 2/56; A61K 9/00; A61K 9/19; A61K 31/19; A61K 31/197; A61K 31/7032; A61K 35/00; A61K 35/744; A61K 35/745; A61K 35/747; A61K 38/08; C12N 1/18; C12N 1/20

(52) U.S. Cl. CPC A61K 36/064; A23L 2/382; A23L 2/56; A61K 9/0095; A61K 9/19; A61K 31/19; A61K 31/197; A61K 31/7032; A61K 35/744; A61K 35/745; A61K 35/747; A61K 38/08; C12N 1/18; C12N 1/20; A23V 2400/113; A23V 2400/121; A23V 2400/123; A23V 2400/125; A23V 2400/147; A23V 2400/151; A23V 2400/173; A23V 2400/175; A23V 2400/427; A23V 2400/51; A61K 2035/115

(57) ABSTRACT

The present disclosure relates to a probiotics-metabolite composition for food and beverage applications. More particularly, the probiotics metabolite composition is a blend comprising whole heat-inactivated yeast in the range of 1-3 billion CFU and whole heat-inactivated lactobacilli in the range of 10-13 billion CFU. The composition can also be a beverage that comprises of fermented plant-only prebiotic fibre, pasteurized co-culture supernatant comprising 2-4 billion CFU of yeast and 5-7 billion CFU of lactobacilli, and plant-only flavouring compound. The said probiotics metabolite blend and beverage can serve to promote digestive health and strengthen the immune system.

### PRIORITY CLAIM

The instant patent application is related to and claims priority from the US provisional patent application entitled, “POSTBIOTICS BLEND FOR FOOD AND BEVERAGE APPLICATIONS”, Patent Application No. 63/634,505, Filed on: 16 Apr. 2024, which is incorporated in its entirety herewith.

### TECHNICAL FIELD

The present disclosure is in the technical field of a probiotic metabolite composition for food and beverage applications. More particularly, the probiotic metabolite composition comprises of short-chain fatty acids (SCFAs), lactate, gamma-aminobutyric acid (GABA), lipoteichoic acid, and pediocin obtained after heat-inactivation of yeast and lactobacilli.

### BACKGROUND OF THE DISCLOSED EMBODIMENT

Probiotic metabolites, also known as postbiotics, are bioactive substances produced as byproducts during the metabolic or fermentation activities of probiotics. These include organic acids, enzymes, peptides, polysaccharides, and other beneficial compounds that support human health.

Probiotics are live microorganisms that require survival and colonization within the gut to confer health benefits such as enhanced digestion and immune function. In contrast, probiotic metabolites are stable, non-living compounds generated by probiotics that directly provide health advantages without relying on gut colonization. Postbiotics are particularly advantageous as they are safer for immunocompromised individuals and find applications in heat-processed foods and pharmaceuticals due to their stability under diverse conditions, presenting a robust alternative to live probiotics.

Unlike their live counterparts, probiotic metabolites do not require refrigeration and are resistant to harsh processing environments, making them well-suited for inclusion in versatile delivery formats like capsules, powders, functional beverages, and snack bars.

In the food and beverage industry, probiotic metabolites offer transformative potential with their multifaceted applications addressing health needs, sustainability goals, and consumer demand for innovative solutions. When incorporated into functional foods and beverages, these non-viable bioactive compounds can improve gut health, enhance immune responses, and regulate metabolism.

By fortifying the gut barrier, reducing inflammation, and modulating the microbiome, probiotic metabolites provide targeted support for managing conditions like irritable bowel syndrome (IBS), diabetes, and dyslipidemia. They also contribute to skin health by alleviating oxidative stress and strengthening the skin barrier, and show promise for mental health applications via the gut-brain axis.

With inherent antimicrobial properties, probiotic metabolites serve as natural, clean-label preservatives, enhancing product shelf life and safety without the need for synthetic additives.

Additionally, they improve sensory experiences in foods by enriching flavors and textures in fermented products, sauces, and snacks.

Offering probiotic-like health benefits without requiring live cultures, probiotic metabolites are especially suitable for health-conscious and sensitive consumer segments.

Sustainable production and adaptability of probiotic metabolites enable the creation of specialized nutrition products, ranging from infant formulas to plant-based alternatives, catering to the increasing demand for eco-friendly and science-driven innovations in the F&B sector.

Probiotic metabolites stand at the forefront of nutraceutical advancements, delivering consistent health benefits without the risks of live bacteria and paving the way for safer, more effective health interventions.

On the other hand, non-alcoholic beverages have experienced growing global demand, driven by health and religious considerations. Unlike regular alcoholic drinks, which may negatively impact gut bacteria balance, low-alcohol or non-alcoholic options offer potential gut health advantages, addressing concerns linked to gut microbiota depletion from alcohol consumption.

Traditional beer production often relies on barley and wheat malts, creating challenges for individuals with gluten sensitivities who seek alternative products.

While numerous probiotic-infused products are commercially available, only a limited selection, such as the Singapore-based beer product “Proficient,” has incorporated live probiotics into beverages. These live-culture products require refrigeration to maintain their viability. Despite confirming the health advantages associated with probiotics, consumers remain cautious about ingesting live organisms.

Examples of probiotic-infused products in the market include “Proficient” and prebiotic sodas like the Canada-based “Crazy D's Sparkling Prebiotic Soda” enriched with dietary fiber.

Currently, there are no commercially available postbiotic-infused products, including postbiotic-containing beers.

The probiotic market has expanded beyond supplements to include fortified products like juices, snacks, and cereals, contributing to its global growth.

However, addressing challenges related to regulations, product stability, sustainability concerns, and consumer education is necessary.

Despite some innovation, probiotic-based beers remain less accessible than mainstream beer options.

Postbiotic products are an emerging segment, with ongoing research exploring their health benefits and diverse applications.

Efforts are underway to develop methods for generating and characterizing probiotic metabolites to incorporate them into products across industries.

Therefore, there is a pressing need to develop a probiotic metabolites composition for food and beverage applications, eliminating refrigeration requirements while retaining the health benefits of probiotics in the form of stable bioactive metabolites, rather than relying on live microorganisms.

### SUMMARY OF THE DISCLOSED EMBODIMENT

According to one embodiment, a composition for probiotics-metabolite blend comprising,
a) a whole heat-inactivated yeast strain; and
b) a whole heat-inactivated bacterial strain.

According to another embodiment, wherein the whole heat-inactivated yeast strain is Saccharomyces cerevisiae, ranging from 1-3 billion CFU; wherein the whole heat-inactivated bacterial strain is Pediococcus pentosaceus, ranging from 10-13 billion CFU.

According to one more embodiment, wherein the yeast strain is selected from Saccharomycopsis fibuligera, Saccharomycopsis malanga, Saccharomyces cerevisiae, and Kluyveromyces marxianus.

According to yet another embodiment, wherein the bacterial strain is selected from Lactobacillus casei, Bifidobacterium, Lactobacillus acidophilus, Lactobacillus delbrueckii subsp. bulgaricus, Lactobacillus helveticus, Pediococcus pentosaceus, Lactobacillus reute, Lactobacillus brevis, Lactobacillus rhamnosus and Lactobacillus johnsonii.

According to another embodiment, wherein the probiotics metabolite blend comprises of GABA, acetate, propionate, lactate, butyrate, lipoteichoic acid and pediocin A; wherein the blend further comprises of polysaccharide-glycopeptide complex, cell-wall components, cytoplasmic extract, cell extract, cell-free supernatant, cytosolic fractions, cell-wall protein fractions, indole-3-aldehyde, secreted proteins, fermentation-derived metabolites, coagulin A, and bacteriocin.

According to yet another embodiment, a method for preparing a probiotics-metabolite blend comprising steps of:
a) growing monocultures of Saccharomyces cerevisiae and Pediococcus pentosaceus in Media One separately, and identifying optimal physiological conditions;
b) co-culturing S. cerevisiae and P. pentosaceus in media one, and identifying optimal co-culture conditions;
c) inoculating 0.5% of S. cerevisiae and P. pentosaceus into the fermentation tank with Media One;
d) co-culturing at conditions with temperature in the range of 26-32° C., pH in the range of 4-6.5, for 24-120 hours;
e) harvesting the fermented culture, centrifuging at 10000 RPM for 10 minutes to obtain a pellet; subjecting the pellet to washing and heat attenuating at 85-90° C. for 45-50 minutes;
f) obtaining the probiotics-metabolite blend; lyophilizing and packing the probiotics-metabolite blend.

According to one more embodiment, wherein Media One comprises of 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate.

According to yet another embodiment, wherein the probiotics metabolite blend comprises of 3 billion CFU of heat-inactivated Saccharomyces cerevisiae and 13 billion CFU of heat-inactivated Pediococcus pentosaceus, further comprising GABA, acetate, propionate, lactate, butyrate, lipoteichoic acid and pediocin A.

According to further embodiment, a composition for probiotics-metabolite beverage comprising,
a) fermented plant only prebiotic fibre;
b) pasteurized co-cultured supernatant of a yeast strain and a bacterial strain;
c) plant only flavouring compound.

According to one embodiment, wherein the fermented prebiotic fibre is from Eleusine coracana; wherein the yeast strain is Saccharomyces cerevisiae; wherein the bacterial strain is Pediococcus pentosaceus; wherein the flavouring compound is from Manilkara zapota.

According to another embodiment, wherein the fermented plant only prebiotic fibre ranges from 8-12%; wherein the pasteurized co-culture supernatant comprises of 2-4 billion CFU of yeast and 5-7 billion CFU of bacteria; wherein the plant only flavouring compound ranges from 2-4%.

According to more embodiments, wherein the plant only prebiotic fibre is selected from selected from pearl millet, foxtail millet, proso millet, finger millet, kodo millet, little millet, sorghum, rice, barley, maize and wheat.

According to one more embodiment, wherein the yeast strain is selected from Saccharomycopsis fibuligera, Saccharomycopsis malanga, Saccharomyces cerevisiae, and Kluyveromyces marxianus.

According to yet another embodiment, wherein the bacterial strain is selected from Lactobacillus casei, Bifidobacterium, Lactobacillus acidophilus, Lactobacillus delbrueckii subsp. bulgaricus, Lactobacillus helveticus, Pediococcus pentosaceus, Lactobacillus reute, Lactobacillus brevis, Lactobacillus rhamnosus and Lactobacillus johnsonii.

According to further embodiment, wherein the plant only flavouring compound is selected from floral, citrus, and tropical fruit flavors.

According to another embodiment, wherein the plant only prebiotic fibre is obtained from Eleusine coracana; wherein the yeast strain is Saccharomyces cerevisiae; wherein the bacterial strain is Pediococcus pentosaceus; wherein the plant only flavouring compound is obtained from Manilkara zapota.

According to one more embodiment, wherein the probiotics-metabolite beverage comprises of GABA, acetate, propionate, lactate, butyrate, lipoteichoic acid and pediocin.

According to another embodiment, the probiotics metabolite beverage contains an Alcohol by Volume (ABV) range of 2.5% to 3.5% and lactic acid levels between 0.5 g/L and 1.5 g/L.

According to various embodiments, a method for preparing a probiotics-metabolite beverage comprising steps of:
a) growing monocultures of Saccharomyces cerevisiae and Pediococcus pentosaceus separately in Media One for identifying the optimal physiological conditions;
b) co-culturing S. cerevisiae and P. pentosaceus in Media One and identifying optimal co-culture conditions;
c) preparing Media Two for fermentation by malting 8-12% of plant only prebiotic fibre obtained from Eleusine coracana at 60-80° C.; hydrolysing by amylase at 50-70° C.; denaturing at 75-100° C.; obtaining a filtrate; vacuum-drying and cooling the filtrate; transferring to the fermentation tank at 15-26° C.;
d) inoculating 0.5% each of S. cerevisiae and P. pentosaceus into the fermentation tank with Media Two;
e) co-culturing at temperature in the range of 26-32° C., pH in the range of 4-6.5, for 24-120 hours;
f) harvesting the fermented culture, centrifuging at 10000 RPM for 10 minutes to obtain a supernatant; blending with 2-4% flavouring compound obtained from Manilkara zapota;
g) pasteurizing the supernatant at 60-80° C. for 15-30 minutes to obtain a probiotic metabolite beverage; and packing of beverage.

According to one embodiment, wherein Media One comprises of 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate.

According to various embodiments, a system for preparing a probiotics-metabolite composition comprising,
a) a data acquisition module for collection of data, wherein the collected data comprises of microorganisms, ingredients for culture media, process parameters for fermentation;
b) a pre-processing module for cleaning and normalizing the collected data to form pre-processed data;
c) a storage module for storing the pre-processed data comprising probiotic organisms and their relative metabolomics, ingredients for culture media, process parameters for fermentation;
d) a machine learning (ML) module for extracting features from the stored pre-proceed data and training a predictive model with the extracted features, wherein the trained predictive model identifies the microorganisms involved in the production of probiotic metabolites; wherein the microorganisms are Saccharomyces cerevisiae and Pediococcus pentosaceus.
e) an inference module for applying the trained predictive model to identify microorganisms, ingredients for culture media, and process parameters for fermentation associated with probiotic metabolite production; and
f) an output module for providing predictions and insights based on the identified microorganism, identified ingredients for culture media, and identified process parameters for fermentation.

According to aspects of present disclosure, wherein the probiotics-metabolite composition is a blend; wherein the blend comprises of heat-inactivated yeast strain Saccharomyces cerevisiae in the range of 1-3 billion CFU and heat-inactivated bacterial strain Pediococcus pentosaceus in the range of 10-13 billion CFU.

According to aspects of present disclosure, wherein the probiotics-metabolite composition is a beverage; wherein the trained predictive model also identifies the ingredients for production of the beverage; wherein the beverage comprises of fermented prebiotic fiber from Eleusine coracana in the range of 8-12%, pasteurized co-culture supernatant comprising 2-4 billion CFU of Saccharomyces cerevisiae and 5-7 billion CFU of Pediococcus pentosaceus and flavouring compound from Manilkara zapota in the range of 2-4%.

According to aspects of present disclosure, wherein the probiotics-metabolite composition comprises of GABA, acetate, propionate, lactate, butyrate, lipoteichoic acid and pediocin.

Several aspects of the disclosed embodiment are described below with reference to examples for illustration. However, one skilled in the relevant art will recognize that the disclosed embodiment can be practiced without one or more of the specific details or with other methods, components, materials and so forth. In other instances, well-known structures, materials, or operations are not shown in detail to avoid obscuring the features of the disclosed embodiment. Furthermore, the features/aspects described can be practiced in various combinations, though only some of the combinations are described herein for conciseness.

### BRIEF DESCRIPTION OF THE DRAWINGS

The foregoing and other objects and features of the disclosed embodiment will become more fully apparent from the following description and appended claims, taken in conjunction with the accompanying drawings. Understanding that these drawings depict only typical embodiments of the disclosed embodiment and are, therefore, not to be considered limiting of its scope, the disclosed embodiment will be described with additional specificity and detail through use of the accompanying drawings briefly described below:

FIG. 1 illustrates the methods involved in the production of probiotic metabolites, according to the aspects of the present disclosure.

FIG. 2 illustrates a flowchart for preparation of a probiotic metabolite blend, according to the aspects of the present disclosure.

FIG. 3 illustrates a flowchart for preparation of a probiotic metabolite beverage, according to the aspects of the present disclosure.

FIG. 4 illustrates the changes in the cell biomass yield with varying temperature (26° C.-36° C.) at pH 6, according to the aspects of the present disclosure.

FIG. 5 illustrates the changes in cell biomass yield with varying pH (4-6.5) at 28° C., according to the aspects of the present disclosure.

FIG. 6 illustrates the co-culture biomass obtained in different capacity fermenters ranging from 15 L-500 L, according to the aspects of the present disclosure.

FIG. 7 illustrates optimization of inoculation time for production of maximum co-culture biomass, according to the aspects of the present disclosure.

FIG. 8 illustrates the yields of cell biomass for yeast, Lactobacilli (LAB), yeast+LAB at 28° C. at pH 6, for 72 hours, according to the aspects of the present disclosure.

FIG. 9 illustrates the yield of yeast and LAB (in billion CFU) obtained in Media One and Media Two, according to the aspects of the present disclosure.

FIG. 10A illustrates the liquid chromatography mass spectrometry product ion mass spectra of, lactate (m/Z 69.09) butyrate (m/Z 62.04), propionate, and lipoteichoic acid (m/Z 503.62 dimer in negative mode) according to the aspects of the present disclosure.

FIG. 10B illustrates the liquid chromatography mass spectrometry product ion mass spectra of gamma-amino butyric acid (m/Z 102, 104) and acetate (m/Z 61, 64), according to the aspects of the present disclosure.

FIG. 10C illustrates the liquid chromatography mass spectrometry product ion mass spectra of pediocin peptide (m/Z 760.49, 827.63), according to the aspects of the present disclosure.

FIG. 11 illustrates a block diagram illustrating the details of a machine learning system, according to the aspects of the disclosed embodiment.

FIG. 12 illustrates a block diagram illustrating the details of a digital processing system, according to the aspects of the present disclosure.

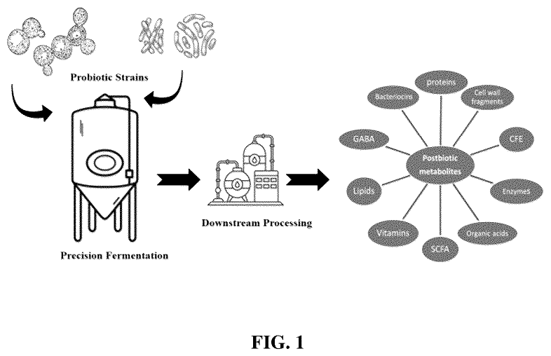

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE DISCLOSED EMBODIMENT

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another. Further, the terms “comprising,” “including,” “having,” and the like, as used with respect to embodiments of the present disclosure, are synonymous. As used herein, the terms “agent,” “component,” or “ingredient” are used interchangeably and refer to a particular item that includes one or more chemical compounds (e.g., a food item from one or more plants that comprise one or more naturally occurring chemical compounds).

The term “component” or “ingredient” as used herein, refers to a particular item that includes one or more chemical compounds, i.e., edible compounds sourced from plants, fungi or algae that comprise one or more naturally occurring chemical compounds.

Alternative embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

As used herein, the singular forms “a”, “an”, and “the” include both singular and plural referents unless the context clearly dictates otherwise. By way of example, “a dosage” refers to one or more than one dosage.

The terms “comprising”, “comprises” and “comprised of” as used herein are synonymous with “including”, “includes” or “containing”, “contains”, and are inclusive or open-ended and do not exclude additional, non-recited members, elements or method steps.

All documents cited in the present specification are hereby incorporated by reference in their totality. In particular, the teachings of all documents herein specifically referred to are incorporated by reference.

Example embodiments of the disclosed embodiments are described with reference to the accompanying figures.

In the drawings, like reference numbers generally indicate identical, functionally similar, and/or structurally similar elements. The drawing in which an element first appears is indicated by the leftmost digit(s) in the corresponding reference number.

### Definitions

The term ‘probiotic metabolites’ blend refers to the metabolites or byproducts produced by probiotic microorganisms during metabolism or fermentation processes. These substances include organic acids, enzymes, peptides, polysaccharides, and other compounds that are beneficial to human health.

The term ‘probiotics’ refer to the live microorganisms known to live symbiotically in the human gut and probiotics sometimes are believed to enhance the presence of other beneficial bacteria within the body bringing in synergistic benefits.

The term ‘fermentation’ involves the breakdown of a substance into simpler components, often facilitated by microorganisms such as yeast and bacteria.

The term ‘co-fermentation’ refers to a technique where two microorganisms are employed to ferment a substrate. The interaction of these can lead to enhanced flavours, nutritional profile and texture. In this embodiment, yeast and lactobacilli were co-fermented to produce a gluten free beverage and a blend comprising probiotic metabolites.

The term ‘Lactobacilli’ are a type of bacteria that produce lactic acid as a by-product of glucose metabolism.

The term “Media One” refers to the fermentation media for production of probiotic metabolite blend. Media One comprises 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate.

The term “Media Two” refers to the fermentation media for production of probiotic metabolite beverage. Media Two comprises of 8-12% of prebiotic fibre obtained from Eleusine coracana.

The term “plant only prebiotic fibre” refer to the carbohydrates obtained from plant sources for fermentation.

The term “plant only flavouring compound” refer to the hops flavours obtained from plant sources (fruits, flower) for preparation of beverage.

The term ‘gluten’ is a protein found in some grains, including wheat, barley, and rye.

The term ‘millets’ are a group of cereal grains that belong to the Poaceae family, commonly known as the grass family. Millet varieties include pearl millet, foxtail millet, proso millet, finger millet, kodo millet, etc.

The term “fermentate” refers to the supernatant obtained by processing the fermented liquid.

The term “heat-inactivation” refers to a de-activation or killed state of a microorganism obtained by subjecting them to high temperatures.

The term “pasteurization” refers to a heat treatment process, named after Louis Pasteur, that kills harmful pathogens and extends shelf life by heating beverages to a specific temperature for a set time, without significantly altering taste or nutritional value.

The term “probiotics-metabolite blend” refers to the pellet obtained by harvesting and processing the fermented liquid comprising a co-culture of yeast and lactobacilli.

The term ‘glycopeptides’ refers to a peptide that contain carbohydrate moieties (glycans) covalently attached to the side chains of the amino acid residues that constitute the peptide.

The term ‘cell wall’ is composed of a network of cellulose microfibrils and cross-linking glycans embedded in a highly cross-linked matrix of pectin polysaccharides in plants and of peptidoglycan network in bacteria.

The term ‘bacteriocins’ refers to a kind of ribosomal synthesized antimicrobial peptide produced by bacteria, which can kill or inhibit bacterial strains closely-related or non-related to produced bacteria, but will not harm the bacteria themselves by specific immunity proteins.

### 1) EMBODIMENTS OF THE DISCLOSURE

The present disclosure relates to a probiotic metabolite composition for food and beverage applications.

In embodiments of the disclosure, two compositions comprising probiotic metabolites are developed; one is a probiotics metabolite blend while the other is a beverage infused with probiotic metabolites. Probiotic microorganisms chosen for the production of these metabolites include yeast and bacterium.

### Yeast and Bacteria for Production of Probiotic Metabolites

The yeast Saccharomyces cerevisiae can produce probiotic metabolites that shown to alter gut microbiome composition and function. Probiotic metabolites such as acetate, propionate, and butyrate are important for gut health and S. cerevisiae can increase the production of such beneficial metabolites.

Additionally, these probiotic metabolites can modulate immune responses and enhance gut barrier integrity.

Lactobacillus species are known for producing various beneficial metabolites during fermentation, such as lactic acid, which contributes to gut health. They can also produce compounds like short-chain fatty acids (SCFAs). These SCFAs play a crucial role in maintaining gut health and modulating the immune system.

Combining probiotic metabolites from S. cerevisiae and Lactobacillus could potentially enhance their individual benefits by promoting a diverse gut microbiome and increasing the variety of beneficial metabolites produced.

### (A) Composition for Probiotic Metabolite Blend

A composition for probiotics-metabolite blend comprising,
a) a whole heat-inactivated yeast strain; and
b) a whole heat-inactivated bacterial strain.

The heat-inactivated yeast strain ranges from 1-3 billion CFU; the heat-inactivated bacterial strain ranges from 10-13 billion CFU; wherein the yeast strain is Saccharomyces cerevisiae; wherein the bacterial strain is Pediococcus pentosaceus.

### Categories of Probiotic Metabolite Blend

### Category I-Yeast Strain

The yeast strain for production of probiotic metabolites was selected from Saccharomycopsis fibuligera, Saccharomycopsis malanga, Saccharomyces cerevisiae, Kluyveromyces marxianus.

In one embodiment, the yeast strain for probiotic metabolites is Saccharomyces cerevisiae.

### Category II-Bacterial Strain

The bacterial strain for production of probiotic metabolites was selected from Lactobacillus casei, Bifidobacterium, Lactobacillus acidophilus, Lactobacillus delbrueckii subsp. bulgaricus, Lactobacillus helveticus, Pediococcus pentosaceus, Lactobacillus reute, Lactobacillus brevis, Lactobacillus rhamnosus, Lactobacillus johnsonii.

In one embodiment, the bacterial strain for production of probiotic metabolites is Pediococcus pentosaceus.

### Preparation of Each Ingredient Category for Probiotics Metabolite Blend

### Category I—Yeast Strain

The yeast cells of Saccharomyces cerevisiae from glycerol stock were inoculated into 10 mL of Media One; wherein Media One comprises 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate.

The yeast culture was incubated for 48 hours at 30° C. with 200 RPM agitation. The yeast growth parameters were optimized for maximum biomass by incubating between 26 and 32° C. for and incubation time of 24-72 hours; and further a lactic acid tolerance study was conducted at 1-5% of lactic acid in growth media.

### Category II—Bacterial Strain

The bacterial cells of Pediococcus pentosaceus from glycerol stock were inoculated into 10 mL of media; wherein Media One comprises 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate. The bacterial culture was incubated for 24 hours at 30° C. with an agitation of 200 RPM. The bacterial growth parameters were optimized for maximum biomass between 26 and 32° C. for an incubation time of 24-72 hours, further an alcohol tolerance study was conducted at 1-10% of alcohol in growth media.

### (B) Method for Production of Probiotic Metabolite Blend

### I) Physiological Conditions for Monocultures of Yeast and Lactobacilli

The yeast strain Saccharomyces cerevisiae and the lactobacilli Pediococcus pentosaceus were grown in media one to identify the best culture conditions individually (202).

One physiological factor at a time method was followed for the study (pH, temperature).

i) Culture Conditions for S. cerevisiae:

Optimum temperature was identified by growing the yeast strain S. cerevisiae in different temperatures ranging from 26-32° C. at pH 6. Optimum pH was determined at optimum temperature 28° C. by maintaining the media pH conditions from 4-6. A growth study was conducted to get maximum biomass at optimized temperature and pH conditions.

ii) Culture Conditions for P. pentosaceus:

Similarly, optimum temperature for the lactobacilli Pediococcus pentosaceus was identified by growing in different temperatures ranging from 26-32° C. at pH 6. Optimum pH was determined at optimum temperature by maintaining the media pH conditions from 4-6. A growth study was conducted to get maximum biomass at optimized temperature and pH conditions.

### II) Physiological Conditions for a Co-Culture Comprising Yeast and Lactobacilli

The yeast strain S. cerevisiae and lactobacilli P. pentosaceus were subjected as a co-culture into media one and grown to identify the optimal co-culturing conditions (204).

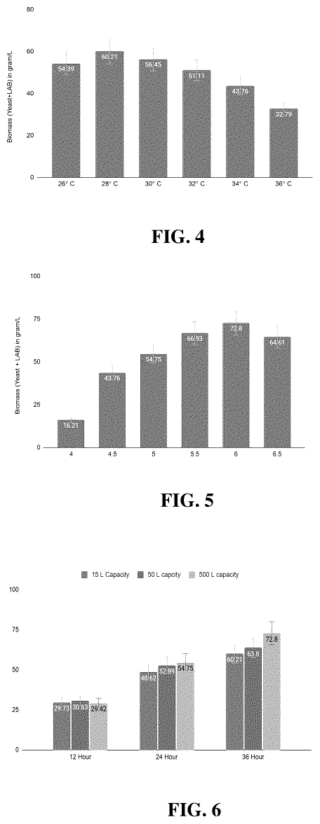

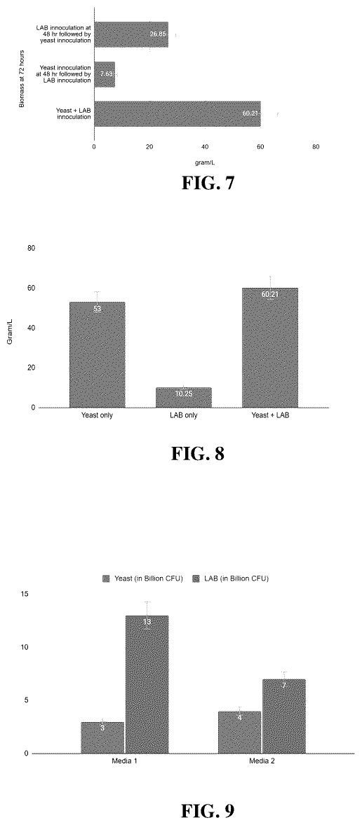

One factor at a time method was followed for the study (pH, temperature). The factors considered were:
a) Optimum temperature was identified by subjecting the co-culture comprising S. cerevisiae and P. pentosaceus to different temperatures ranging from 26-36° C. at pH 6 as illustrated in Table 1 and FIG. 4.
b) Optimum pH for the co-culture was determined at optimum temperature 28° C. by maintaining the media pH conditions from 4-6 as illustrated in Table 2 and FIG. 5.
c) Growth study of the co-culture was performed to get maximum biomass at optimized temperature and pH conditions as illustrated in Table 3 and FIG. 6.
d) The co-culture inoculation time was studied by employing three different inoculation strategies wherein first strategy involved inoculating yeast followed by lactobacilli post 48 hours; wherein the second strategy involved inoculating lactobacilli followed by yeast post 48 hours and the third strategy involved inoculating both yeast and lactobacilli together (zero-hour inoculation) as illustrated in Table 4 and FIG. 7.

The physiological conditions identified and considered for fermenting co-culture comprising yeast and lactobacilli for production of probiotic metabolite blend were temperature ranging from 26-32° C., pH ranging from 4-6 (206).

### III) Fermentation of Co-Culture Comprising Yeast and Lactobacilli for Production of Probiotic Metabolites

Media one comprising 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate was subjected into the fermentation tank (208).

Optical density (OD) of the yeast and bacterial strains was measured using the colorimeter at 600 nm. Initial OD of both strains were maintained at 0.4 for inoculation.

Further, 0.5% each of the individually grown yeast strain S. cerevisiae and lactobacilli P. pentosaceus were subjected as a co-culture into the fermentation tank. Zero-hour inoculation was practiced wherein the yeast and lactobacilli were cultured together at the same time. The strains were co-fermented under the conditions, temperature: 26-32° C., pH: 4-6.5, for a time period of 24-120 hours (210).

IV) Downstream Processing to Obtain the Probiotics Metabolites from the Co-Culture

After 120 hours, the fermented liquid was harvested and centrifuged at 10000 RPM for 10 minutes to obtain the pellet (212).

### V) Heat-Attenuation of Pellet

The pellet obtained after processing the fermented liquid was washed and subjected to heat-inactivation/attenuation at 85-90° C. for 45-50 minutes for obtaining a probiotics metabolite blend. The strains present in pellet were inactivated by adopting a heat attenuation technique to preserve the generated probiotics metabolites (214).

### Advantages of Heat-Attenuating the Co-Culture:

i) Preservation of Metabolites: Once microorganisms are inactivated, they can no longer consume or degrade the metabolites they have produced. This helps preserve the desired compounds and prevents changes to the product's composition.
ii) Termination of by-product Formation: Active microorganisms might continue producing unwanted by-products or altering the fermentation environment. Inactivation stops these processes, ensuring the purity of the harvested metabolites.
iii) Facilitates Downstream Processing: Inactivating microbes simplifies subsequent steps like purification and extraction. It prevents contamination from live cells and reduces the risk of microbial spoilage during product handling.

### VI) Lyophilization of the Pellet

The pellet was further subjected to lyophilization/freeze drying for removal of moisture.

### Advantages of Lyophilizing the Pellet:

i) Preservation of Viability: Lyophilization stabilizes the biological activity of microorganisms in the co-culture, ensuring their viability over extended periods without refrigeration. This is particularly useful for maintaining the integrity of probiotics, enzymes, or other sensitive components.
ii) Extended Shelf Life: By removing moisture, the process prevents microbial growth and chemical degradation, significantly extending the shelf life of the co-culture. This makes storage and transportation more convenient and cost-effective.
iii) Ease of Reconstitution: Lyophilized co-cultures can be easily rehydrated by adding water or a suitable buffer, restoring their functionality and activity almost instantly.
iv) Reduced Weight and Volume: The freeze-dried product is lightweight and compact, making it easier to package and transport, especially in bulk quantities.
v) Enhanced Stability: Lyophilization minimizes the risk of oxidation and other environmental factors that could compromise the quality of the co-culture during storage.

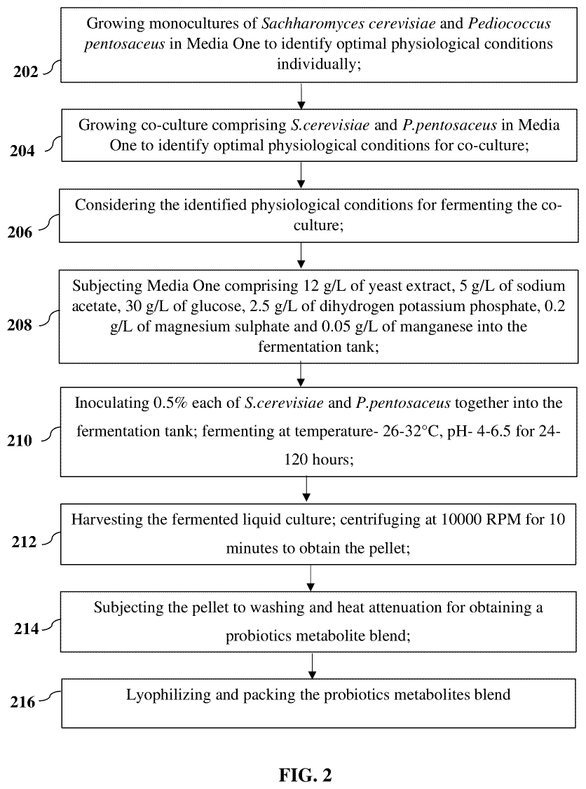

The lyophilized/freeze dried pellet was obtained in a powdered form to be processed for packaging (216) as illustrated in (FIG. 2).

The obtained pellet comprises a co-culture of 1-3 billion CFU of heat-inactivated Saccharomyces cerevisiae and 10-13 billion CFU of heat-inactivated Pediococcus pentosaceus. Co-culturing these strains yields seven probiotic metabolites and other essential components; wherein the metabolites comprise of GABA, acetate, propionate, lactate, butyrate, lipoteichoic acid and pediocin A; wherein the essential components comprise of polysaccharide-glycopeptide complex, cell-wall components and cytoplasmic extract, cell extract, cell-free supernatant, cytosolic fractions, cell-wall protein fractions, indole-3-aldehyde, secreted proteins, fermentation-derived metabolites, coagulin A, and bacteriocin.

### (C) Composition for Probiotics Metabolite Beverage

A composition for probiotics-metabolite beverage comprising,
a) fermented plant only prebiotic fibre;
b) pasteurized co-culture supernatant of yeast strain and bacterial strain;
c) plant only flavouring compound.

### Categories of Probiotic Metabolite Beverage

### Category I—Plant Only Prebiotic Fibre

The plant only prebiotic fibre for preparation of beverage was selected from pearl millet, foxtail millet, proso millet, finger millet, kodo millet, little millet, sorghum, rice, barley, maize and wheat.

In one embodiment, the prebiotic fibre for preparation of beverage was obtained from Eleusine coracana.

Eleusine coracana, commonly known as finger millet, is a versatile cereal crop with immense potential for beverage production. Its rich nutritional profile, including high levels of calcium, iron, and dietary fiber, makes it an excellent base for health-focused drinks. Finger millet can be fermented to make beverages, enhancing its nutritional value and promoting gut health. Additionally, its bioactive compounds contribute to antioxidant properties, making it suitable for functional beverages.

### Category II—Yeast Strain

The yeast strain for production of probiotic metabolites was selected from Saccharomycopsis fibuligera, Saccharomycopsis malanga, Saccharomyces cerevisiae, Kluyveromyces marxianus.

In one embodiment, the yeast strain for probiotic metabolites is Saccharomyces cerevisiae.

### Category III—Bacterial Strain

The bacterial strain for production of probiotic metabolites was selected from Lactobacillus casei, Bifidobacterium, Lactobacillus acidophilus, Lactobacillus delbrueckii subsp. bulgaricus, Lactobacillus helveticus, Pediococcus pentosaceus, Lactobacillus reute, Lactobacillus brevis, Lactobacillus rhamnosus, Lactobacillus johnsonii.

In one embodiment, the bacterial strain for production of probiotic metabolites is Pediococcus pentosaceus.

### Category IV—Plant Only Flavouring Compound

The plant flavouring compound for preparation of beverage was selected from hops including floral, citrus, and tropical fruit flavors.

In one embodiment, the flavouring compound for preparation of the beverage was obtained from Manilkara zapota.

Manilkara zapota, commonly known as sapodilla or chikoo, is a tropical fruit with sweet, caramel-like flavor, making it suitable for beverage preparation. Its high sugar content and unique taste profile allow it to be used as a natural sweetener and flavor enhancer in juices, smoothies, and fermented drinks. The bioactive compounds phenolic acids, flavonoids (such as anthocyanins and flavanols), and tannins, which provide its sweet, caramel-like taste and mild astringency. Additionally, sugars, ascorbic acid (vitamin C), and aromatic compounds enhance its natural sweetness and aroma,

Sapodilla also contains bioactive compounds such as polyphenols and minerals, contributing to antioxidant and anti-inflammatory properties, which can elevate the nutritional value of beverages.

### Preparation of Each Ingredient Category for Probiotics Metabolite Beverage

### Category I—Plant-Only Prebiotic Fibre

8-12% of plant only prebiotic fibre obtained from Eleusine coracana flour was cooked and malted at 60-80° C. and hydrolysed by the enzyme amylase (0.02-0.04 g/Kg of the substrate). The obtained filtrate was vacuum dried, using a 25-50-micron mesh filter.

The techniques hydrolysis and malting occur simultaneously. Malting happens naturally with help of intrinsic enzymatic degradation of starches, however effective hydrolysis of flour is attained by combining malting with hydrolysis by additional amylase treatment. This combined treatment produces maximum sugars that serve as a substrate during fermentation.

A denaturation step at 75-100° C. is necessary to denature the amylase enzyme to make sure the enzyme is completely inactivated.

### Category II—Yeast Strain

The yeast cells of Saccharomyces cerevisiae from glycerol stock were inoculated into 10 mL of Media One; wherein Media One comprises 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate.

The yeast culture was incubated for 48 hours at 30° C. with 200 RPM agitation. The yeast growth parameters were optimized for maximum biomass by incubating between 26 and 32° C. for and incubation time of 24-72 hours; and further a lactic acid tolerance study was conducted at 1-5% of lactic acid in growth media.

### Category III—Bacterial Strain

The bacterial cells of Pediococcus pentosaceus from glycerol were inoculated into 10 mL of Media One; wherein Media One comprises 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate. The bacterial culture was incubated for 24 hours at 30° C. with an agitation of 200 RPM. The bacterial growth parameters were optimized for maximum biomass between 26 and 32° C. for an incubation time of 24-72 hours, further an alcohol tolerance study was conducted at 1-10% of alcohol in growth media.

### Category IV—Plant Only Flavouring Compound

The plant only flavouring compound was obtained from Manilkara zapota and was prepared in the range of 2-4% for incorporation into the probiotics metabolite beverage.

Addition of flavouring compounds is not a mandatory blending step as the intrinsic components of the ingredient system itself adds flavour to the beverage.

### Significance of Plant Only Prebiotic Fibre and Flavouring Compound

### i) Plant Only Prebiotic Fibres

Plant only prebiotic fibres comprise of mono/disaccharides (carbohydrate) source for the microorganisms to act upon. These serve as adjunct to fermentation substrates, gluten free substrate, dietary fiber (soluble). The plant only prebiotic fibres are employed to enhance mouth feel, crispiness, reduce haziness of beverage and impart a lighter color to the beverage. These are digestible, convertible and fermentable sugars. 6-(hydroxymethyl) oxane-2,3,4,5-tetrol, α-D-glucopyranosyl-(1→4)-D-glucose, etc are examples of bioactive compounds, and chemical names found in the plant only substrates. Alternatively, simple sugars like glucose, maltose, cane sugar, etc also can be employed during fermentation.

### ii) Plant Only Flavouring Compounds

The plant only flavouring compounds selected from hops flavours were employed to impart taste and odour to the beverage.

### (D) Method for Production of Probiotic Metabolite Beverage

### I) Physiological Conditions for Monocultures of Yeast and Lactobacilli

The yeast strain Saccharomyces cerevisiae and the lactobacilli Pediococcus pentosaceus were grown in Media One to identify the best culture conditions individually (302).

One physiological factor at a time method was followed for the study (pH, temperature).

i) Culture Conditions for S. cerevisiae:

Optimum temperature was identified by growing the yeast strain S. cerevisiae in different temperatures ranging from 26-32° C. at pH 6. Optimum pH was determined at optimum temperature by maintaining the media pH conditions from 4-6.5. A growth study was conducted to get maximum biomass at optimized temperature and pH conditions.

ii) Culture Conditions for P. pentosaceus:

Similarly, optimum temperature for the lactobacilli Pediococcus pentosaceus was identified by growing in different temperatures ranging from 26-32° C. at pH 6. Optimum pH was determined at optimum temperature by maintaining the media pH conditions from 4-7. A growth study was conducted to get maximum biomass at optimized temperature and pH conditions.

### II) Physiological Conditions for a Co-Culture Comprising Yeast and Lactobacilli

The yeast strain S. cerevisiae and lactobacilli P. pentosaceus were subjected as a co-culture into media one and grown to identify the best culture conditions for co-culture (304).

One factor at a time method was followed for the study (pH, temperature). The factors considered were:
a) Optimum temperature was identified by subjecting the co-culture comprising S. cerevisiae and P. pentosaceus to different temperatures ranging from 26-36° C. at pH 6 as illustrated in Table 1 and FIG. 4.
b) Optimum pH for the co-culture was determined at optimum temperature 28° C. by maintaining the media pH conditions from 4-6.5 as illustrated in Table 2 and FIG. 5.
c) Growth study of the co-culture was performed to get maximum biomass at optimized temperature and pH conditions as illustrated in Table 3 and FIG. 6.
d) The co-culture inoculation time was studied by employing three different inoculation strategies wherein first strategy involved inoculating yeast followed by lactobacilli post 48 hours; wherein the second strategy involved inoculating lactobacilli followed by yeast post 48 hours and the third strategy involved inoculating both yeast and lactobacilli together (zero-hour inoculation) as illustrated in Table 4 and FIG. 7.

The physiological conditions identified and considered for fermenting co-culture comprising yeast and lactobacilli for production of probiotic metabolite beverage were temperature ranging from 26-32° C., pH ranging from 4-6.5 (306).

### III) Fermentation of the Co-Culture Comprising Yeast and Lactobacilli for Production of Probiotic Metabolite Beverage

A method for preparing a probiotics metabolite beverage composition, wherein the method comprises steps of:

8-12% of plant only prebiotic fibre obtained from Eleusine coracana flour was cooked and malted at 60-80° C. and hydrolysed by the enzyme amylase (0.02-0.04 g/kg of the substrate). The obtained filtrate was vacuum dried, using a 25-50-micron mesh filter. The filtrate was cooled and transferred to the fermentation tank at 15-26° C. This filtrate prepared from plant-based flour was considered as Media Two (308).

Optical density (OD) of the yeast and bacterial strains was measured using the colorimeter at 600 nm. Initial OD of both strains were maintained at 0.4 for inoculation.

Further, 0.5% each of the individually grown yeast strain S. cerevisiae and lactobacilli P. pentosaceus were subjected as a co-culture into the fermentation tank. Zero-hour inoculation was practiced wherein the yeast and lactobacilli were cultured together at the same time. The strains were co-fermented under the conditions, temperature: 26-32° C., pH: 4-6, for a time period of 24-120 hours (310).

IV) Downstream Processing to Obtain the Probiotics Metabolite Beverage from the Co-Culture

After 120 hours, the fermented liquid was harvested and centrifuged at 10000 RPM for 10 minutes to extract the supernatant (312).

2-4% of plant only flavouring compound obtained from Manilkara zapota was added to the supernatant (optional) (314).

### V) Pasteurization of the Supernatant

Finally, the supernatant was subjected to pasteurization at 60-80° C. for 15-30 minutes. Pasteurization of supernatant was performed for:
i) Microbial Inactivation: Both yeast and bacteria, including the desirable ones, are killed or rendered inactive due to the heat treatment. This halts fermentation processes, ensuring no further microbial activity takes place.
ii) Shelf Stability: Pasteurization prevents spoilage by eliminating unwanted microbes, enhancing the beverage's shelf life.
iii) Controlled Composition: The process locks in the current balance of flavors, metabolites, and textures created by fermentation, ensuring consistency in the product.
iv) Nutritional Preservation: While the heat may degrade certain heat-sensitive nutrients or bioactive compounds, the bulk of the beverage's nutritional value remains intact.

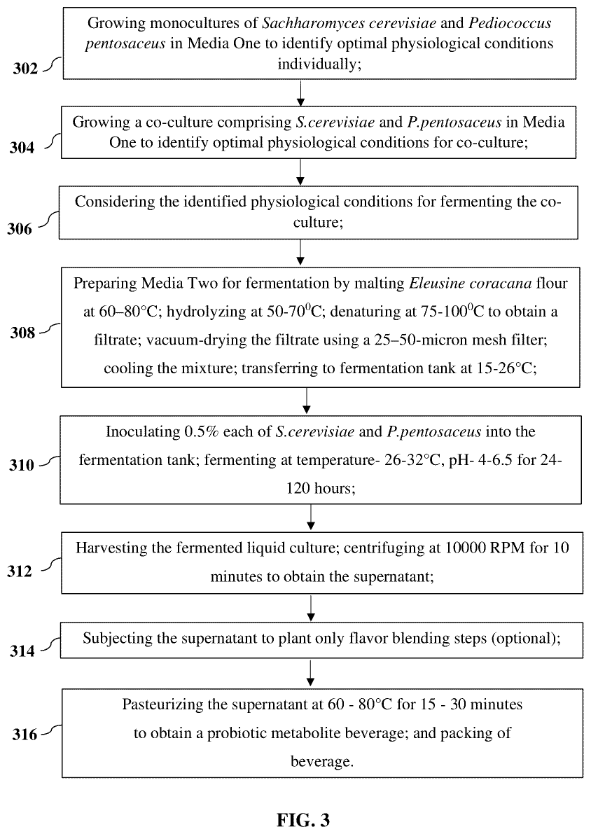

The pasteurized supernatant was said to be the probiotic metabolite beverage and was processed for packaging (316) as illustrated in (FIG. 3).

The obtained probiotics metabolite beverage comprises of fermented prebiotic fibre ranging from 8-12%, pasteurized co-culture supernatant comprises of 2-4 billion CFU of yeast and 5-7 billion CFU of bacteria and flavouring compound ranging from 2-4%. The co-culture of these strains yields seven probiotic metabolites; wherein the metabolites include GABA, acetate, propionate, lactate, butyrate, lipoteichoic acid and pediocin A.

### 2) EXPERIMENTAL EVIDENCES

### (I) Production of Probiotic Metabolites in Monoculture and Co-Culture

Saccharomyces cerevisiae is a probiotic yeast that can produce metabolites that support gut health, immunity, and metabolic functions. It majorly includes manno-proteins, beta glucans, organic acids like acetate and lactate. Pediococcus pentosaceus is a species of Lactobacillus which produces metabolites like lactate, butyrate, acetate, propionate, lipoteichoic acid, pediocin, gamma amino butyric acid which can provide gut health, antimicrobial, immune-boosting, and gut-brain axis benefits. The mono-culture of these two strains cannot produce all these metabolites in detectable quantities. However, a co-culture of these two microbes Saccharomyces cerevisiae and Pediococcus pentosaceus creates a synergistic relationship, where each species benefits from the other's metabolic by-products. The yeast breaks down complex polysaccharides, making more fermentable sugars available for SCFA synthesis by P. pentosaceus whereas P. pentosaceus thrives in acidic conditions and can utilize ethanol and yeast metabolites to enhance acetate and lactate production. Also, the low GABA production in native yeast is enhanced under acidic growth conditions which will be created by P. pentosaceus. The presence of yeast cell wall components (mannoproteins, β-glucans) may also induce stress responses in the lactobacilli, leading to higher pediocin synthesis. Moreover, the lactate produced by lactobacilli will act as precursor for butyrate production by gut microbiota and beta glucan of yeast acts as prebiotic fibre which also will serve as source for butyrate production by gut microbiota. With this hypothesis in premise, a co-culture of Saccharomyces cerevisiae and Pediococcus pentosaceus was optimised at a condition of 28° C. and pH 6 where a minimum 13 billion CFU of LAB and 3 billion CFU of yeast was generated to produce a pool of seven metabolites including acetate, butyrate, propionate, LTA, GABA, lactate and pediocin. It is not possible to have all these metabolites from any single organism and hence the co-culture is a novel approach to have the aforementioned metabolites conferring a host of benefits when consumed. All the aforementioned metabolites were detected by LCMS analysis in the final product.

### (II) Physiological Parameters for Co-Culture

In general, the yeast and Lactobacillus strains do not grow together because yeast growth is being inhibited by the high amount of lactic acid produced or Lactobacillus cannot survive in the presence of alcohol produced by yeast. Optimisation of co-culture was done across a range of temperature and pH to arrive at a condition which yielded optimal cell biomass. It is evident from the data sets provided with respect to temperature and pH conditions. An optimised condition of 28° C. and pH 5.5 was finalized from the trials which produced the highest cell biomass among the trials. The optimal growth of both the organisms Saccharomyces cerevisiae and Pediococcus pentosaceus in the co-culture is also evident from the CFU count conducted post the harvest of each batch of fermentation. 13 billion CFU of LAB and 3 billion CFU of yeast is a desirable cell biomass in order to have the above-mentioned metabolites to be present in the final product. If not as per optimised conditions, co-culturing at a temperature above 30° C., lactobacilli will outgrow yeast and would make the metabolite profile completely different. This will limit the production of SCFA and beta glucans and therefore in the final product. Vice versa, co-culturing at temperatures below 30° C. will allow yeast to outgrow lactobacilli which will make the final product devoid of LTA, lactate and pediocin. Also, the presence of pediocin cannot be detected if lactobacilli alone is grown and in contrary higher pediocin production was observed due to co-culture of lactobacilli and yeast. Thus, it is crucial to maintain the co-culture as per optimised conditions to have all the beneficial probiotic metabolites in the final product as desired.

(III) Significance of Co-Fermentation of Lactobacilli with Yeast

Fermenting plant only prebiotic fibres with both yeast and lactobacilli (a type of lactic acid bacteria) is done to obtain the advantages of co-fermentation. The process of co-fermentation can add complexity to the flavor profile of the final product while the interaction between yeast and lactobacilli can result in unique aromas and flavours, contributing to a more diverse sensory experience. Lactobacilli can also influence the mouth feel of the beverage, contributing to a smoother or fuller texture additionally, the bacteria also produce lactic acid, which can impart a tangy or sour taste to the beverage. The production of lactic acid also lowers the pH of the fermentation environment which can inhibit the growth of harmful bacteria and contribute to preservation. Lactobacilli are considered probiotic bacteria, potentially offering health benefits such as improved gut health and digestion. This bacterial genus includes various strains like Lactobacillus brevis and Lactobacillus plantarum, are relatively tolerant to moderate levels of alcohol compared to many other bacteria and controlled conditions, including pH, temperature, and alcohol levels, is crucial to support their activity. Hence the disclosure here, involves a strain of Lactobacilli, which survives a significantly higher level of alcohol environment, that makes it possible to have the benefits of lactic acid in the product.

### (IV) Synergistic Effect of the Co-Culture Strains

i) Probiotic Metabolites from Yeast

Beta-glucans, mannan-oligosaccharides and SCFA's (short-chain fatty acids) are the major metabolites for positive effects on gut health, including supporting the intestinal barrier and providing an energy source for colon cells. These probiotic metabolites might be used in functional foods, supplements, or pharmaceuticals to support immune function, gut health, and overall well-being.

ii) Probiotic Metabolites from Lactobacilli

Lactic Acid, exopolysaccharides (EPS) and bacteriocins are the lactobacilli derived metabolites for supporting gut health, modulating the immune system, improving digestive function, and potentially offering protection against certain diseases.

iii) Effects of Probiotic Metabolites from Different Organisms

Utilizing probiotic metabolites from different microbial strains in combination, whether in form of product formulations, fermented foods, or supplements, may offer a more comprehensive approach to supporting gut health, immune function, and overall well-being. This approach capitalizes on the unique properties of various probiotic metabolites, potentially amplifying their individual benefits when combined together.

Synergism of ingredients and organisms in the proprietary fermentation process for obtaining a probiotics metabolite beverage

Brewing malt extract of plant only prebiotic fibres as wort for co-fermentation using the strains of yeast and lactobacilli has resulted in a flavor with optimum mouth feel and body as that of a commercial beer, similar to porter style of beer. Moreover, it has also been identified that the synergy of the two different organisms involved in the co-fermentation. The co-fermentation conditions were standardized to optimum conditions to have the end product with 2.5-3.5% ABV and 0.5 g/L to 1.5 g/L lactic acid that provides a typical characteristic taste profile of a typical sour beer contributed by both the organisms involved in the fermentation.

### (V) Synergy of Metabolites & Functionality of the Probiotic Metabolites

The co-culture of yeast and lactobacilli yields a pool of metabolites including short Chain Fatty Acids (SCFAs), LTA, GABA, lactate and pediocin. It is not possible to have all these metabolites from any single organism and hence the co-culture is a novel approach to have the aforementioned metabolites conferring a host of benefits when consumed.

SCFAs comprising acetate, butyrate, propionate in the gut are involved in gastrointestinal physiology, immune functions, microbiome alterations and also in gut-brain axis interactions.

Lipotechoic acid (LTA) is an important surface component of Gram-positive bacteria that executes cellular functional roles. LTA can be used as dietary supplements, or therapeutic formulations to enhance gut health, manage inflammation, and support immune functions.

GABA is an important probiotic metabolite considered an inhibitory neurotransmitter that helps in reducing stress and anxiety while enhancing sleep quality.

Lactate can positively influence the levels of butyrate and propionate levels, as lactate acts as a precursor via CoA transferase pathway in gut microbes. Lactate, plays a vital role in maintaining gut pH by influencing the microbial ecosystem and the biochemical environment of the gut. It contributes to creating an acidic environment that inhibits the growth of pH-sensitive pathogenic bacteria, thus promoting a healthier microbial balance.

Pediocin, a bacteriocin produced by certain strains of Pediococcus species, serves as a potent antimicrobial agent to support gut health and immune function. Pediocin PA-1, a peptide has antimicrobial efficacy making it suitable to be an alternative bio-preservative agent in the food industry as it has proven efficacy against both spoilage and pathogenic organisms, including L. monocytogenes, Enterococcus faecalis, S. aureus, and Clostridium perfringens.

The disclosure is a probiotic metabolite blend comprising of short-chain fatty acids (SCFAs), lactate, gamma-aminobutyric acid (GABA), lipoteichoic acid, and pediocin to attain a synergistic advantage by targeting multiple health pathways. SCFAs strengthen the gut barrier, regulate inflammation, and provide energy for colonocytes, while lactate enhances SCFA production and maintains an acidic environment that inhibits pathogens. This acidic milieu further boosts the antimicrobial efficacy of pediocin, a potent bacteriocin, providing robust defense against harmful microbes. GABA, known for its gut-brain axis modulation, complements the anti-inflammatory effects of SCFAs and teichoic acids, which also support immune balance and reduce chronic inflammation. Together, these components not only enhance gut health and microbiota balance but also promote systemic benefits, including improved immunity, stress resilience, and metabolic regulation, making the formulation a holistic approach to well-being.

### 3) EXAMPLE EMBODIMENTS

### Example 1: Temperature Conditions for Production of Probiotic Metabolites in Co-Culture

TABLE 1
TEMPERATURE CONDITIONS FOR PRODUCTON OF
PROBIOTIC METABOLITES IN CO-CULTURE

| | Temperature | | | | | |
|---|---|---|---|---|---|---|
| | 26° C. | 28° C. | 30° C. | 32° C. | 34° C. | 36° C. |
| Biomass | 54.39 | 60.21 | 56.45 | 51.11 | 43.76 | 32.79 |
| (Yeast + L | | | | | | |
| AB) in | | | | | | |
| gram/L | | | | | | |

The co-culture comprising 0.5% of yeast and 0.5% of lactobacilli (LAB) was grown in Media One to identify the optimal temperature conditions. The co-culture was grown in temperatures ranging from 26° C.-36° C. for 24-120 hours and the wet cell weight (WCW) of biomass (Yeast+LAB) was calculated. The highest biomass was obtained when the co-culture was grown at 28° C. as it is a favourable condition for yeast to grow, which contributes to the biomass majorly along with required growth of LAB. The yield of biomass was reduced at 36° C. which favours growth of LAB when compared to yeast (FIG. 4).

### Example 2: pH Conditions for Production of Probiotic Metabolites in Co-Culture

TABLE 2
pH CONDITIONS FOR PRODUCTON OF PROBIOTIC
METABOLITES IN CO-CULTURE

| | pH | | | | | |
|---|---|---|---|---|---|---|
| | 4 | 4.5 | 5 | 5.5 | 6 | 6.5 |
| Biomass | 16.21 | 43.76 | 54.75 | 66.93 | 72.8 | 64.61 |
| (Yeast + L | | | | | | |
| AB) in | | | | | | |
| gram/L | | | | | | |

The co-culture comprising 0.5% of yeast and 0.5% of Lactobacilli (LAB) was grown in Media One to identify the optimal pH conditions. The co-culture was grown in pH ranging from 4-6 for 24-120 hours and the wet cell weight (WCW) of biomass (Yeast+LAB) was calculated. The highest biomass of co-culture was obtained at pH 6 hence it was considered optimal for fermentation (FIG. 5).

### Example 3: Co-Culture Biomass Obtained in Different Capacity Fermentors

TABLE 3
CO-CULTURE BIOMASS OBTAINED IN
DIFFERENT CAPACITY FERMENTORS

| | Biomass in gram/L (WCW) | | |
|---|---|---|---|
| | 15 L | 50 L | 500 L |
| | Capacity | Capacity | Capacity |
| 12 Hour | 29.73 | 30.63 | 29.42 |
| 24 Hour | 48.62 | 52.89 | 54.75 |
| 36 Hour | 60.21 | 63.8 | 72.8 |

The comparative Table 3 represents the trend of co-culture biomass growth at different time points of the process in fermentors of different capacities. The co-culture comprising 0.5% of yeast and 0.5% of lactobacilli (LAB) was grown in media one in three different fermenters with capacities of 15 L, 50 L and 500 L. The data reflects on the consistency of the biomass obtained in different capacities of fermenters. The co-culture biomass was found to increase as the capacity of the fermenter increased (FIG. 6).

### Example 4: Inoculation Time for Production of Probiotic Metabolites

TABLE 4
INOCULATION TIME FOR PRODUCTON
OF PROBIOTIC METABOLITES

Biomass at 72 hours

| | LAB | Yeast | |
|---|---|---|---|
| | inoculation | inoculation | |
| | at 48 hr | at 48 hr | Yeast + |
| | followed | followed | LAB |
| | by yeast | by LAB | inoculation |
| | inoculation | inoculation | together |
| gram/L | 26.85 | 7.63 | 60.21 |
| (WCW) | | | |

The data given in Table 4 signifies the inoculation time of the organisms and its correlation with the yield of biomass. Three types of inoculation strategies were followed to obtain the best possible probiotic metabolites. In the first strategy, lactobacilli (LAB) was inoculated into the fermentation tank after 48 hours of inoculation of yeast whereas in the second strategy, yeast was inoculated after 48 hours of LAB inoculation. In the third strategy, a zero-hour inoculation method was adopted wherein LAB and yeast were inoculated together into the fermentation tank (FIG. 7).

### Results & Interpretations:

The first inoculation strategy yielded a co-culture biomass of 26.85 g/L while the second strategy yielded a biomass of 7.63 g/L and the third type yielded a biomass of 60.21 g/L. It was inferred that zero-hour inoculation was the best practice to obtain maximum co-culture biomass.

### Example 5: Biomass Yield in Monocultures & Co-Cultures

TABLE 5
BIOMASS YIELD FOR PRODUCTON
OF PROBIOTIC METABOLITES

| Growth at 30 °C and pH 5.5 | Gram/L (WCW) |
|---|---|
| Yeast only | 53 |
| LAB only | 10.25 |
| Yeast + LAB | 60.21 |

The data given in Table 5 shows the difference in biomass yield when the organisms were cultured individually (monoculture) and together (co-culture) conditions in Media One. The data reflects optimised conditions that facilitate the co-culture yielding an optimal biomass of 60.21 g/L (WCW when compared to the wet cell weight of the monocultures of yeast and LAB (FIG. 8).

### Example 6: Culture Media for Production of Probiotic Metabolites

TABLE 6
CULTURE MEDIA FOR PRODUCTON OF PROBIOTIC
METABOLITES IN BLEND AND BEVERAGE

| | Yeast (in Billion | LAB (in Billion |
|---|---|---|
| | CFU) | CFU) |
| Media One | 3 | 13 |
| (Commercial media) | | |
| Media Two | 4 | 7 |
| (Optimized media) | | |

The data given in Table 6 represents the difference in growth of yeast and LAB in commercial media vs optimised media in billion CFU (FIG. 9).

Media One (Commercial media) comprising 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate was utilized for obtaining the probiotics metabolite blend comprising a co-culture of 3 billion CFU of yeast and 13 billion CFU of LAB

Media Two (Optimized media) comprising 8-12% of fermented plant only prebiotic fibre obtained from Eleusine coracana was utilized for obtaining the probiotics metabolite beverage comprising a co-culture of 4 billion CFU of yeast and 7 billion CFU of LAB.

### Example 7: Tolerance of Lactic Acid Bacteria in Ethanol Media

TABLE 7
REPRESENTATION OF OD VALUES OBSERVED
IN 2, 4, 5% ETHANOL MEDIA AT 24 HOURS
AND 48 HOURS OF INCUBATION

| | OD | | | | | | |
|---|---|---|---|---|---|---|---|
| | value | | | | | | |
| | without | | | | | | |
| | EtOH | | | | | | |
| | Trial 1, | 2% ETOH | | 4% ETOH | | 5% ETOH | |
| Sample | 2 & 3 | 24 h | 48 h | 24 h | 48 h | 24 h | 48 h |
| Trail 1 | 7.14 | 6.76 | 6.36 | 6.87 | 4.84 | 8.26 | 4.99 |
| Trail 2 | 6.23 | 6.58 | 6.46 | 6.83 | 6.09 | 8.47 | 4.42 |
| Trail 3 | 5.42 | 6.44 | 6.84 | 4.85 | 5.92 | 4.88 | 4.16 |

The above OD values in Table 7 represent that the growth of LAB is not inhibited at up to 5% EtOH in the media for 48 hours of time period and it is evident as the OD values of 3 different colonies growth is comparable to that from the media not containing EtOH used as control media. From the above it can be inferred that this LAB strain grows normally in media containing up to 5% EtOH similarly as it grows in media without EtOH.

### Example 8: Tolerance of Yeast in Media Containing Lactic Acid

The lactic acid tolerance of the yeast was performed by culturing the yeast in presence of 1% lactic acid in both Media One and Media Two.

In Media One, at the co-culturing condition, the lactic acid produced was 0.288% while in Media Two, the lactic acid produced was 0.78-0.861%.

From this, it can be inferred that the lactic acid produced during the fermentation was within the limit thereby confirming the tolerance of yeast in lactic acid containing media.

### Example 9: LC-MS Analysis for Probiotic Metabolite Blend and Beverage

The co-culture pellet obtained after fermentation was subjected to heat attenuation and freeze drying for preparation of fine powder of probiotic metabolites. The obtained probiotics metabolite powder was subjected to ethanol extraction and then run through LCMS and the spectra obtained was analysed against the library. Similarly, the co-culture supernatant was pasteurized, and run through LCMS and the spectra obtained was analysed against the library.

### Results:

Both the samples, co-culture pellet and supernatant yielded the seven metabolites; wherein the metabolites include GABA, acetate, propionate, lactate, butyrate, lipoteichoic acid and pediocin A.

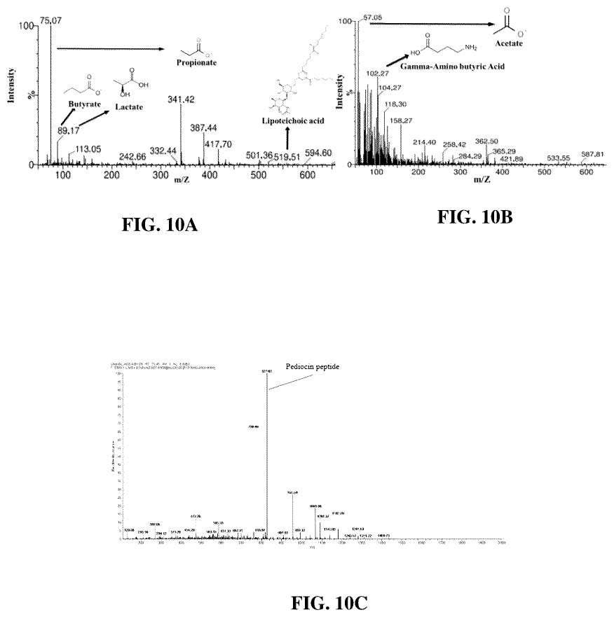

Lactate is an organic acid with molecular mass of 90.08 g/mol. A spectrum was found in the negative mode of ionization and in a dehydrated form it matches the mass of lactate. Butyrate is a short chain fatty acid, molecular mass is 87.10 g/mol and was found in negative mode of ionization and its dehydrated form matches the mass of butyrate. Propionic acid is an organic acid with molecular mass of 74.08 g/mol. A spectrum was found in the negative mode of ionization and in a dehydrated form it matches the mass of propionate (FIG. 10A).

Lipotechoic acid is a cell wall polymer with a molecular mass of 1032 g/mol and a spectrum of 503.62 g/mol as mass was found in negative mode of ionization and as a dimer it matches the mass of lipoteichoic acid (FIG. 10A).

Gamma amino butyric acid (GABA) is a neurotransmitter and also chemically an amino acid with a molecular mass of 103.12 g/mol. Spectral responses of 102.12 g/mol and 104.27 g/mol mass were found in positive mode of ionization and with protonation added and it matches the mass of GABA. Acetate is a monocarboxylic acid with a molecular mass of 59.04 g/mol. Spectral responses of 61.04 and 62.04 2 g/mol as mass were found in both positive and negative mode of ionization and with protonation added and it matches the mass of acetate (FIG. 10 B).

Pediocin is a peptide and analysed separately by trypsin digestion of the sample and peptide spectra analysis against proteome discoverer 2.5 version using the database downloaded from Uniport. The peptide mentioned in FIG. 10C showed similarity with pediocin compounds present in the database and confirmed the presence of it in the sample.

### 4) MACHINE LEARNING SYSTEM FOR PREPARATION OF THE PROBIOTICS METABOLITE COMPOSITION

A machine learning system for preparing probiotics metabolite composition, wherein the system comprises of:
a) a data acquisition module for collection of data, wherein the collected data comprises of microorganisms, plant only ingredients for culture media, process parameters for fermentation;
b) a pre-processing module for cleaning and normalizing the collected data to form pre-processed data;
c) a storage module for storing the pre-processed data comprising probiotic organisms and their relative metabolomics, plant only ingredients for culture media, process parameters for fermentation;
a) a machine learning (ML) module for extracting features from the stored pre-proceed data and training a predictive model with the extracted features, wherein the trained predictive model identifies the microorganisms involved in the production of probiotic metabolites; wherein the microorganisms are Saccharomyces cerevisiae and Pediococcus pentosaceus.
d) an inference module for applying the trained predictive model to identify microorganisms, plant only ingredients for culture media, and process parameters for fermentation associated with probiotic metabolite production; and
e) an output module for providing predictions and insights based on the identified microorganism, identified plant only ingredients for culture media, and identified process parameters for fermentation.

The mentioned modules a-f of the machine learning system are illustrated in (FIG. 11) and described in the following embodiment.

### a) Data Acquisition Module

The data acquisition module is for collection of data, wherein the collected data comprises of microorganisms, plant only ingredients for culture media, process parameters for fermentation, optimization for probiotic metabolites production (1102).

### b) Pre-Processing Module

The pre-processing module is for cleaning and normalizing the collected data to form pre-processed data (1104).

### c) Storage Module

The storage module is for storing the pre-processed data comprising probiotic organisms and their relative metabolomics, plant only ingredients and plant only flavour systems and their relative functionalities (1106).

### d) Machine Learning (ML) Module

The machine learning (ML) module is configured for extracting features from the stored pre-proceed data and training a predictive model with the extracted features, wherein the trained predictive model identifies the organisms involved in the production of probiotic metabolites (1108).

### Predictive Model

The trained predictive model determines a combination of organisms for production of probiotic metabolites, wherein the combination is of a yeast and a bacterium; wherein the yeast is selected from Saccharomycopsis fibuligera, Saccharomycopsis malanga, Saccharomyces cerevisiae, Kluyveromyces marxianus, wherein the bacterium is selected from Lactobacillus casei, Bifidobacterium, Lactobacillus acidophilus, Lactobacillus delbrueckii subsp. bulgaricus, Lactobacillus helveticus, Pediococcus pentosaceus, Lactobacillus reute, Lactobacillus brevis, Lactobacillus rhamnosus, Lactobacillus johnsonii.

The trained predictive model considers the process parameters including physical parameters (temperature, pH, RPM), nutritional parameters (carbon source, nitrogen source and their proportion) and micro nutrition (salts and metal ions) in the fermenter for optimal growth of the co-culture for probiotic metabolite production, wherein the trained predictive model also identifies a heat inactivation technique to attenuate the live co-culture and freeze drying for preparation of fine powder of probiotic metabolites.

According to one embodiment, the probiotics-metabolite composition is a blend; wherein the blend comprises of a whole heat-inactivated yeast strain Saccharomyces cerevisiae in the range of 1-3 billion CFU and a whole heat-inactivated bacterial strain Pediococcus pentosaceus in the range of 10-13 billion CFU.

According to one more embodiment, the probiotics-metabolite composition is a beverage; wherein the trained predictive model also identifies the ingredients for production of the beverage; wherein the beverage comprises of fermented prebiotic fiber from Eleusine coracana in the range of 8-12%, pasteurized co-culture supernatant comprising 2-4 billion CFU of Saccharomyces cerevisiae and 5-7 billion CFU of Pediococcus pentosaceus and flavouring compound from Manilkara zapota in the range of 2-4%.

According to various embodiments, the probiotics-metabolite composition yields seven metabolites including GABA, acetate, propionate, lactate, butyrate, lipoteichoic acid and pediocin.

### e) Inference Module

The inference module is configured for applying the trained predictive model to identify microorganisms, plant only ingredients for culture media, and process parameters for fermentation associated with probiotic metabolite production (1112). The microorganisms identified for production of probiotic metabolites are Saccharomyces cerevisiae and Pediococcus pentosaceus.

The optimized parameters for the production of probiotic metabolites are temperature in the range of 28-32° C., pH in the range of 4-6.5.

### f) Output Module

The output module is configured for providing predictions and insights based on the identified microorganism, identified plant only ingredients for culture media, and identified process parameters for fermentation (1114).

### 5) HARDWARE

### Digital Processing System

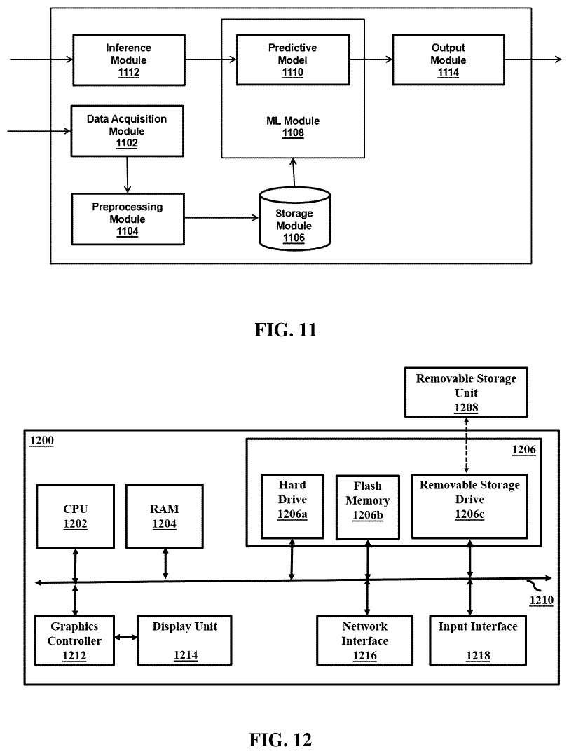

(FIG. 12) is a block diagram illustrating the details of a digital processing system (1200) in which various aspects of the present disclosure are operative by execution of appropriate execution modules, firmware or hardware components.

Digital processing system (1200) may correspond to each of user system: local system or remote and server noted above. Digital processing system may contain one or more processors (such as a central processing unit (CPU) (1202)), random access memory (RAM) (1204), secondary memory (1206), graphics controller (GPU) (1208), display unit (1214), network interfaces like (WLAN) (1216), and input interfaces (1212).

CPU (1202) executes instructions stored in RAM (1204) to provide several features of the present disclosure. CPU (1202) may contain multiple processing units, with each processing unit potentially being designed for a specific task.

Alternatively, CPU (1202) may contain only a single general purpose processing unit. RAM (1204) may receive instructions from secondary/system memory (1210).

Graphics controller (GPU) (1208) generates display signals (e.g., in RGB format) to primary display unit (1214) based on data/instructions received from CPU (1202). Primary display unit contains a display screen (1214) (e.g., monitor, touchscreen) to display the images defined by the display signals. Input interfaces (1212) may correspond to a keyboard, a pointing device (e.g., touch-pad, mouse), a touchscreen, etc. which enable the various inputs to be provided. Network interface (1216) provides connectivity to a network (e.g., using Internet Protocol), and may be used to communicate with other connected systems. Network interface (1216) may provide such connectivity over a wire (in the case of TCP/IP based communication) or wirelessly (in the case of WIFI, Bluetooth based communication).

Secondary memory (1206) may contain hard drive (mass storage) (1206a), flash memory (1206b), and removable storage drive (1206c). Secondary memory (1206) may store the data (e.g., the specific requests sent, the responses received, etc.) and executable modules, which enable the digital processing system (1200) to provide several features in accordance with the present disclosure.

Some or all of the data and instructions may be provided on a removable storage unit (SD card) (1208), and the data and instructions may be read and provided by removable storage drive (1206c) to CPU (1202). Floppy drive, magnetic tape drive, CD-ROM drive, DVD Drive, Flash memory, removable memory chip (PCMCIA Card, EPROM) are other examples of such removable storage drive (1206c).

Removable storage unit (1208) may be implemented using storage format compatible with removable storage drive (1206c) such that removable storage drive (1206c) can read the data and instructions. Thus, removable storage (1206c) unit includes a computer readable storage medium having stored therein executable modules and/or data. However, the computer (or machine, in general) readable storage medium can be in other forms (e.g., non-removable, random access, etc.). CPU (1202) may retrieve the executable modules, and execute them to provide various features of the present disclosure described above.

### 6) IMPORTANT ATTRIBUTES OF THE DISCLOSED EMBODIMENT

Synergism of the strains: The disclosure involves a process for fermenting a co-culture comprising of a strain of yeast and a Lactobacillus which synergistically grow under optimized conditions, thereby yielding two end products. The products comprise of probiotic metabolites from two different organisms which is in contrast from the existing products in the market which are generated using a single microorganism.

Zero-waste process: The disclosed method is a zero-waste process with complete utilization of the fermentate (supernatant) and pellet generated from fermentation; the fermentate is a beverage infused with probiotic metabolites whereas the pellet is a dry blend of the probiotic metabolites.

Novelty: The present disclosure is a novel approach to have the benefits of probiotics in the form of probiotic metabolites in beverage which do not have the probiotic (live culture) inclusion directly, however the benefits that probiotics can confer are covered by augmenting specific molecules (probiotic metabolites) into the product.

Growing Health Consciousness: Probiotic metabolites are an alternative or complementary approach to probiotics, providing health benefits without the need to introduce live microorganisms into the gut. Understanding the specific mechanisms and effects of different probiotic metabolites can lead to the development of targeted therapies or dietary interventions for promoting gut health and overall well-being.

Increased shelf life and less maintenance: Unlike probiotics, which require careful storage conditions to maintain their viability, probiotic metabolites are stable and do not require refrigeration, thus enhances the shelf life of the beverage. Their non-viable nature reduces the risk of contamination, simplifying manufacturing processes and ensuring product safety.

Regulatory requirement: Probiotic metabolites might have a smoother regulatory pathway compared to live probiotics since they are non-living components. This simplifies the regulatory process for manufacturers. These metabolites can be easily incorporated into various beverage formulations without concerns about the survival or stability of live microorganisms during processing or storage. Probiotic metabolites can provide similar health benefits to probiotics, such as supporting gut health, modulating the immune system, and potentially offering anti-inflammatory effects.

Ethical issues: Some consumers may prefer probiotic metabolites over live probiotics due to concerns about the survival of live cultures in products, making probiotic metabolites more appealing. Formulating beverages with probiotic metabolites might simplify the manufacturing process compared to incorporating live probiotics, reducing complexities associated with handling and processing.

Enhanced gut health: Probiotic metabolites tend to be more resistant to processing conditions like heat, acidity, or other factors that might compromise the viability of live probiotics. Production costs might be lower for beverages containing probiotic metabolites compared to those with live probiotics due to reduced handling and storage requirements.

Mimicking nature of the beverage: The disclosure includes probiotic metabolite infused beverage with additional benefits of being a gluten free variant and giving a classical sour beer taste profile. The combination of plant only prebiotic fibres brings an unique flavor and well-balanced characteristics, imparting nutty, earthy, or slightly sweet flavors, contributing to the overall complexity of the beer's taste profile, lightening the body of the beer, giving it a crisper and light mouthfeel, enhancing clarity and giving it a clean, neutral flavor, contributing to a smoother and more refreshing taste.

Products featuring probiotic metabolites can capitalize on the growing consumer interest in gut health without the limitations associated with live cultures, potentially widening the market reach. Therefore, the disclosed embodiment leverages the advantage of probiotic metabolites in beverages providing an avenue to create a product that promotes gut health and cater to consumer preferences for functional and health-focused beverages.

### 7) USES, APPLICATIONS AND BENEFITS OF THE DISCLOSED EMBODIMENT

As an ingredient usage in food application, the recommended dosage for probiotic metabolite blend is usually 20-200 mg. The blend in dry powder can be re-hydrated and consumed as such. The blend can also be added to products like milk, beverages, ice cream, gummy, bakery, etc. The dose can be optimized based on the CFU count of probiotics in the finished probiotic metabolite blend product. For example, 20 mg of probiotic metabolite blend powder may contain up to 20 billion probiotic cells.

The disclosed probiotics metabolite blend is intended for the following applications:

Beverage application: The probiotics metabolite blend can be formulated into different types of beverages with natural fruit flavours. The disclosure offers a gluten-free alternative to traditional beer, providing added nutritional benefits from dietary fibers and polyphenols.

Nutraceutical application: The said blend composition of intended nutraceutical application is comprised of probiotic metabolites generated in the process, plant-based gums such as flaxseed gum, fenugreek gum, psyllium husk, as well as dietary fibers like inulin, fructo oligosaccharides and corn fiber.

Cosmetic application: The probiotics metabolite blend of intended cosmetic application can be formulated with water; plant-based butters such as salt butter, shea butter, and mango butter as the oil phase; humectants like glycerin; stabilizers and thickeners such as xanthan gum, guar gum, lecithin, and methyl cellulose; and preservatives including sodium benzoate, vitamin E acetate, lemongrass oil, and lavender oil, along with probiotic metabolites generated in the process.

Metabolites produced by probiotic organisms offer significant potential in food and beverage applications as these compounds contribute to gut health by modulating the microbiota and strengthening the gut barrier, thus aiding digestion and reducing inflammation.

According to the further aspects of the disclosure, with these immunomodulatory effects, probiotic metabolites improve immune function via anti-inflammatory and antioxidant potential, thereby alleviating chronic diseases. Moreover, some of these metabolites can enhance nutrient absorption and extend product shelf life through antimicrobial activity. As a result, incorporating postbiotics into food and beverage formulations presents an opportunity to create functional products that support overall health and meet consumer demand for natural, minimally processed options.

### 8) BEST MODE TO PRACTICE THE DISCLOSED EMBODIMENT

Best mode to practice the present disclosure is by commercializing probiotic metabolite blend as it's a product with increasing consumer acceptance for individuals choosing healthier lifestyles. Interestingly, all the multinational brands are entering the space of the probiotic metabolite market, looking at the encouraging growth rate of the probiotic metabolite supplements market and consumer acceptance of healthy alternative choice of products.

The probiotic metabolite blend can also be infused in beverages like beers, energy drinks, sodas and other carbonated beverages. It can also be added to conventional dairy-based products such as yoghurts, flavored milks, ice creams. As the blend is heat stable, it can also be incorporated into baked goods or confectionery.

On the other hand, beer itself is a 800-billion-dollar market, non-alcoholic and low alcohol beverages are picking their spot in the market with an impressive growth rate which is an encouraging factor for this kind of product. The probiotic metabolite beverage composition can be commercialised as a gluten-free alternative to traditional beer, providing added nutritional benefits from dietary fibers and polyphenols.

Merely for illustration, only representative number/type of graph, chart, block, and sub-block diagrams were shown. Many environments often contain many more block and sub-block diagrams or systems and sub-systems, both in number and type, depending on the purpose for which the environment is designed.

While specific embodiments of the disclosure have been shown and described in detail to illustrate the inventive principles, it will be understood that the disclosure may be embodied otherwise without departing from such principles.

It should be understood that the figures and/or screen shots illustrated in the attachments highlighting the functionality and advantages of the disclosed embodiment are presented for example purposes only. The disclosed embodiment is sufficiently flexible and configurable, such that it may be utilized in ways other than that shown in the accompanying figures.

It should be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A composition for probiotics-metabolite blend comprising,
a) a whole heat-inactivated yeast strain; and
b) a whole heat-inactivated bacterial strain.

2. The composition as claimed in claim 1, wherein the whole heat-inactivated yeast strain is Saccharomyces cerevisiae, ranging from 1-3 billion CFU; wherein the whole heat-inactivated bacterial strain is Pediococcus pentosaceus, ranging from 10-13 billion CFU.

3. The composition as claimed in claim 1, wherein the yeast strain is selected from Saccharomycopsis fibuligera, Saccharomycopsis malanga, Saccharomyces cerevisiae, and Kluyveromyces marxianus.

4. The composition as claimed in claim 1, wherein the bacterial strain is selected from Lactobacillus casei, Bifidobacterium, Lactobacillus acidophilus, Lactobacillus delbrueckii subsp. bulgaricus, Lactobacillus helveticus, Pediococcus pentosaceus, Lactobacillus reute, Lactobacillus brevis, Lactobacillus rhamnosus and Lactobacillus johnsonii.

5. The composition as claimed in claim 1, wherein the probiotics metabolite blend comprises of GABA, acetate, propionate, lactate, butyrate, lipoteichoic acid and pediocin A; wherein the blend further comprises of polysaccharide-glycopeptide complex, cell-wall components, cytoplasmic extract, cell extract, cell-free supernatant, cytosolic fractions, cell-wall protein fractions, indole-3-aldehyde, secreted proteins, fermentation-derived metabolites, coagulin A, and bacteriocin.

6. A method for preparing a probiotics-metabolite blend comprising steps of:
a) growing monocultures of Saccharomyces cerevisiae and Pediococcus pentosaceus in Media One separately, and identifying optimal physiological conditions;
b) co-culturing S. cerevisiae and P. pentosaceus in media one, and identifying optimal co-culture conditions;
c) inoculating 0.5% of S. cerevisiae and P. pentosaceus into the fermentation tank with Media One;
d) co-culturing at conditions with temperature in the range of 26-32° C., pH in the range of 4-6.5, for 24-120 hours;
e) harvesting the fermented culture, centrifuging at 10000 RPM for 10 minutes to obtain a pellet; subjecting the pellet to washing and heat attenuating at 85-90° C. for 45-50 minutes;
f) obtaining a probiotics-metabolite blend; lyophilizing and packing the probiotics-metabolite blend.

7. The method as claimed in claim 6, wherein Media One comprises of 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate.

8. The method as claimed in claim 6, wherein the probiotics metabolite blend comprises of 3 billion CFU of heat-inactivated Saccharomyces cerevisiae and 13 billion CFU of heat-inactivated Pediococcus pentosaceus, further comprising GABA, acetate, propionate, lactate, butyrate, lipoteichoic acid and pediocin A.

9. A composition for probiotics-metabolite beverage comprising,
a) fermented plant only prebiotic fibre;
b) pasteurized co-culture supernatant of a yeast strain and a bacterial strain;
c) plant only flavouring compound.

10. The composition as claimed in claim 9, wherein the fermented prebiotic fibre is from Eleusine coracana; wherein the yeast strain is Saccharomyces cerevisiae; wherein the bacterial strain is Pediococcus pentosaceus; wherein the flavouring compound is from Manilkara zapota.

10. The composition as claimed in claim 9, wherein the fermented plant only prebiotic fibre ranges from 8-12%; wherein the pasteurized co-culture supernatant comprises of 2-4 billion CFU of yeast and 5-7 billion CFU of bacteria; wherein the plant only flavouring compound ranges from 2-4%.

11. The composition as claimed in claim 9, wherein the plant only prebiotic fibre is selected from selected from pearl millet, foxtail millet, proso millet, finger millet, kodo millet, little millet, sorghum, rice, barley, maize and wheat.

12. The composition as claimed in claim 9, wherein the yeast strain is selected from Saccharomycopsis fibuligera, Saccharomycopsis malanga, Saccharomyces cerevisiae, and Kluyveromyces marxianus.

13. The composition as claimed in claim 9, wherein the bacterial strain is selected from Lactobacillus casei, Bifidobacterium, Lactobacillus acidophilus, Lactobacillus delbrueckii subsp. bulgaricus, Lactobacillus helveticus, Pediococcus pentosaceus, Lactobacillus reute, Lactobacillus brevis, Lactobacillus rhamnosus and Lactobacillus johnsonii.

14. The composition as claimed in claim 9, wherein the plant only flavouring compound is selected from floral, citrus, and tropical fruit flavors.

15. The composition as claimed in claim 9, wherein the probiotics-metabolite beverage comprises of GABA, acetate, propionate, lactate, butyrate, lipoteichoic acid and pediocin; wherein the beverage has an Alcohol by Volume (ABV) ranging from 2.5% to 3.5% and lactic acid levels between 0.5 g/L and 1.5 g/L.

16. A method for preparing a probiotics-metabolite beverage, comprising steps of:
a) growing monocultures of Saccharomyces cerevisiae and Pediococcus pentosaceus separately in Media One for identifying the optimal physiological conditions;
b) co-culturing S. cerevisiae and P. pentosaceus in Media One and identifying optimal co-culture conditions;
c) preparing Media Two for fermentation by malting 8-12% of plant only prebiotic fibre obtained from Eleusine coracana at 60-80° C.; hydrolysing by amylase at 50-70° C.; denaturing at 75-100° C.; obtaining a filtrate; vacuum-drying and cooling the filtrate; transferring to the fermentation tank at 15-26° C.;
d) inoculating 0.5% each of S. cerevisiae and P. pentosaceus into the fermentation tank with Media Two;
e) co-culturing at temperature in the range of 26-32° C., pH in the range of 4-6.5, for 24-120 hours;
f) harvesting the fermented culture, centrifuging at 10000 RPM for 10 minutes to obtain a supernatant; blending with 2-4% flavouring compound obtained from Manilkara zapota;
g) pasteurizing the supernatant at 60-80° C. for 15-30 minutes to obtain a probiotic metabolite beverage; and packing of beverage.

17. The method as claimed in claim 16, wherein Media One comprises of 12 g/L of yeast extract, 5 g/L of sodium acetate, 30 g/L of glucose, 2.5 g/L of dihydrogen potassium phosphate, 0.2 g/L of magnesium sulphate and 0.05 g/L of manganese sulphate.

18. A system for preparing a probiotics-metabolite composition comprising,
a) a data acquisition module for collection of data, wherein the collected data comprises of microorganisms, ingredients for culture media, process parameters for fermentation;
b) a pre-processing module for cleaning and normalizing the collected data to form pre-processed data;
c) a storage module for storing the pre-processed data comprising probiotic organisms and their relative metabolomics, ingredients for culture media, process parameters for fermentation;
d) a machine learning (ML) module for extracting features from the stored pre-proceed data and training a predictive model with the extracted features, wherein the trained predictive model identifies the microorganisms involved in the production of probiotic metabolites; wherein the microorganisms are Saccharomyces cerevisiae and Pediococcus pentosaceus.
e) an inference module for applying the trained predictive model to identify microorganisms, ingredients for culture media, and process parameters for fermentation associated with probiotic metabolite production; and
f) an output module for providing predictions and insights based on the identified microorganism, identified ingredients for culture media, and identified process parameters for fermentation.

19. The system as claimed in claim 18, wherein the probiotics-metabolite composition is a blend; wherein the blend comprises of a whole heat-inactivated yeast strain Saccharomyces cerevisiae in the range of 1-3 billion CFU and a whole heat-inactivated bacterial strain Pediococcus pentosaceus in the range of 10-13 billion CFU.

20. The system as claimed in claim 18, wherein the probiotics-metabolite composition is a beverage; wherein the trained predictive model also identifies the ingredients for production of the beverage; wherein the beverage comprises of fermented prebiotic fiber from Eleusine coracana in the range of 8-12%, pasteurized co-culture supernatant comprising 2-4 billion CFU of Saccharomyces cerevisiae and 5-7 billion CFU of Pediococcus pentosaceus and flavouring compound from Manilkara zapota in the range of 2-4%.

21. The system as claimed in claim 18, wherein the probiotics-metabolite composition comprises of GABA, acetate, propionate, lactate, butyrate, lipoteichoic acid and pediocin.
